(19) United States
(12) Patent Application Publication
(10) Pub. No.: US 2011/0044952 A1
(43) Pub. Date: Feb. 24, 2011

(54) AMPLIFICATION OF CANCER-SPECIFIC ONCOLYTIC VIRAL INFECTION BY HISTONE DEACETYLASE INHIBITORS

(75) Inventors: John Cameron Bell, Ottawa (CA); John Hiscott, Montreal (CA); Hesham Abdelbary, Toronto (CA); Thi Lien-Anh Nguyen, Montreal (CA); Jean-Simon Diallo, Ottawa (CA)

Correspondence Address: FULBRIGHT & JAWORSKI L.L.P., 600 CONGRESS AVE., SUITE 2400, AUSTIN, TX, 78701, US

(73) Assignee: Ottawa Health Research Institute, Ottawa, Ontario (CA)

(21) Appl. No.: 12/745,030

(22) PCT Filed: Nov. 26, 2008
(86) PCT No.: PCT/CA08/02090

§ 371 Date: Oct. 22, 2010

Related U.S. Application Data
(60) Provisional application No. 60/996,605, filed on Nov. 27, 2007.

(51) Int. Cl. A61K 35/76; A61P 35/00

(52) U.S. Cl. 424/93.6

(57) ABSTRACT

The invention provides methods for treating cancer cells in a host by infecting the cancer cells with one or more strains of oncolytic virus, in conjunction with treating the host with an amount of an HDI that is effective to augment the cancer-cell-specific oncolytic infection.

### FIELD

The invention is in the field of cancer treatment, particularly oncolytic viral therapies.

### BACKGROUND

A wide variety of oncolytic viruses have been used in preclinical and clinical cancer therapies (see Parato et al., 2005; Bell et al, 2003; Everts and van der Poel, 2005; Ries and Brandts, 2004). For example, an improved therapeutic response has been reported in patients suffering from squamous cell cancer who receive a combination of oncolytic virus therapy and chemotherapy, compared to patients who receive chemotherapy alone (Xia et al., 2004). Oncolytic viruses that have been selected or engineered to productively infect tumor cells include adenovirus (Xia et al., 2004; Wakimoto et al., 2004); reovirus; herpes simplex virus 1 (Shah, et al., 2003); Newcastle disease virus (NDV; Pecora, et al., 2002); vaccinia virus (Mastrangelo et al., 1999; US 2006/0099224); coxsackievirus; measles virus; vesicular stomatitis virus (Stojdl, et al., 2000; Stojdl, et al., 2003); influenza virus; myxoma virus (Myers, R. et al., 2005). For example, EP 1218019, US 2004/208849, US 2004/115170, WO 2001/019380, WO 2002/050304, WO 2002/043647 and US 2004/170607 disclose oncolytic viruses, such as Rhabdovirus, picornavirus, and vesicular stomatitis virus (VSV), in which the virus may exhibit differential susceptibility, particularly for tumor cells having low PKR activity. WO 2005/007824 discloses oncolytic vaccinia viruses and their use for selective destruction of cancer cells, which may exhibit a reduced ability to inhibit the antiviral dsRNA dependent protein kinase (PKR) and increased sensitivity to interferon. WO 2003/008586 similarly discloses methods for engineering oncolytic viruses, which involve alteration or deletion of a viral anti-PKR activity. WO 2002/091997, US 2005/208024 and US 2003/77819 disclose oncolytic virus therapies in which a combination of leukocytes and an oncolytic virus in suspension may be administered to a patient. WO 2005/087931 discloses selected Picornavirus adapted for lytically infecting a cell in the absence of intercellular adhesion molecule-1 (ICAM-1). WO 2005/002607 discloses the use of oncolytic viruses to treat neoplasms having activated PP2A-like or Ras activities, including combinations of more than one type and/or strain of oncolytic viruses, such as reovirus. US 2006/18836 discloses methods for treating p53-negative human tumor cells with the Herefordshire strain of Newcastle disease virus. WO 2005/049845, WO 2001/053506, US 2004/120928, WO 2003/082200, EP 1252323 and US 2004/9604 disclose herpes viruses such as HSV, which may have improved oncolytic and/or gene delivery capabilities.

In many instances, oncolytic viral vectors have been administered by intratumoral injection, such as vectors based on vaccinia virus, adenovirus, reovirus, newcastle disease virus, coxsackievirus and herpes simplex virus (HSV) (Shah et al., 2003; Kaufman, et al. 2005; Chiocca et al., 2004; Harrow et al., 2004; Mastrangelo et al., 1999). In metastatic disease, a systemic route of delivery for oncolytic viruses may be desirable, for example by intravenous administration (Reid et al., 2002; Lorence et al., 2003; Pecora et al., 2002; Lorence et al., 2005; Reid et al., 2001; McCart et al., 2001).

Histone deacetylase inhibitors (HDIs) are compounds that inhibit the enzymatic activity of histone deacetylase. The following documents, incorporated herein by reference, disclose a variety of HDIs: AU 2001/18768 B2, AU 2002/327627 B2, U.S. Pat. No. 6,897,220, US 0039850, U.S. Pat. No. 6,541,661, U.S. Pat. No. 7,288,567, U.S. Pat. No. 7,253,204, AU 2001/283925 B2, U.S. Pat. No. 7,282,608, U.S. Pat. No. 7,250,514, U.S. Pat. No. 7,169,801, U.S. Pat. No. 7,154,002, U.S. Pat. No. 6,495,719, U.S. Pat. No. 7,057,057, U.S. Pat. No. 7,214,831, U.S. Pat. No. 7,191,305, U.S. Pat. No. 7,126,001, U.S. Pat. No. 7,205,304, EP 12068086 B1, U.S. Pat. No. 6,511,990, U.S. Pat. No. 7,244,751, AU 2002/246053 B2, AU 2000/68416 B2, U.S. Pat. No. 7,091,229, U.S. Pat. No. 6,638,530, EP 1501508 B1, EP 1656348 B1, EP 1358168 B1, U.S. Pat. No. 7,067,551, AU 2001/282129 B2, U.S. Pat. No. 6,552,065, US 683384, EP 1301184 B1, EP 1318980 B1, U.S. Pat. No. 6,960,685, U.S. Pat. No. 6,888,027, EP 1335898 B1, U.S. Pat. No. 7,183,298, U.S. Pat. No. 7,135,493, U.S. Pat. No. 6,825,317, U.S. Pat. No. 6,656,905.

HDIs have been introduced as chemotherapeutic compounds capable of inducing growth arrest, differentiation and/or apoptosis of cancer cells ex vivo, as well as in vivo in tumor-bearing animal models (Kelly, 2005; Minucci, 2006; Taplin, 2007; Mehnert, 2007). Several different classes of HDIs are now undergoing clinical trials as anti-tumor agents (Moradei, 2005; Dokmanovic, 2005; Johnstone, 2002; Marks, 2004; Taddei, 2005; Glaser, 2007). Vorinostat/SAHA (suberoylanilide hydroxamic acid) was the first FDA-approved HDI for the treatment of cutaneous T-cell lymphoma (Mann, 2007; Mann, 2007). The HDI MS-275 has been used clinically in multiple Phase I trials with leukemia patients (Gojo et al., 2007).

### SUMMARY

In one aspect, the invention relates to the demonstration that HDIs may be used therapeutically in conjunction with an oncolytic virus so as to amplify the oncolytic infection of a cancer cell, preserving or augmenting the selectivity of the viral infection for cancer cells over non-cancer cells in a host.

In various aspects, the invention provides methods for treating cancers. The methods may involve infecting cancer cells with an amount of one or more strains of oncolytic virus. The virus will generally be selected to be effective to cause a lytic infection in cancer cells. In alternative embodiments, one or more strains of an oncolytic virus may be used in methods of the invention, simultaneously or successively. A virus may for example be selected from the group consisting of: vesicular stromatitis virus (VSV), vaccinia virus, and herpes simplex virus, such as HSV1. In some embodiments, the virus may be a cancer cell selective oncolytic virus that is susceptible in the cancer cell to an inhibitory interferon response. In such embodiments, a HDI may be selected for use with the virus so that the HDI attenuates the inhibitory interferon response in the cancer cell. In alternative embodiments, HDIs may for example be selected from the following: MS-275, SAHA, VPA, and PXD-101.

In alternative embodiments, the oncolytic virus may be administered to the host systemically, such as intravenously, or intratumorally to infect the tumor. The oncolytic virus and a HDI may, for example, be co-administered. Alternative hosts amenable to treatments in accordance with the invention may include animals, mammals and humans.

In various aspects, the invention accordingly provides for the use of one or more HDIs to increase the susceptibility of a tumor or cancer cell to oncolytic viral infection.

### BRIEF DESCRIPTION OF THE DRAWINGS

FIG. 1: illustrates that combined treatment with VSV and HDIs increases viral replication in various cancer cell lines. Cell lines were either non-treated (NT) or treated with MS-275, SAHA, VPA, or PXD-101 for 24 hours and then infected with VSV-d51-GFP at MOI 10⁻⁴. GFP expression was monitored at 35 hours post-infection (Panel A). The results of cell viability assays are illustrated in Panel B.

FIG. 2: illustrates that combined treatment with VSV and HDIs induces caspase-mediated apoptosis in prostate cancer cells. PC3 cells were either non-treated (NT) or pre-treated with MS-275 or SAHA for 24 hours and then infected or not-infected with VSV-d51 at 0.1 MOI. As shown in Panel A, at 96 hours post-infection, PC3 cells treated with the VSV/HDIs combination presented the morphology of dead cells. As shown in Panel B, the percentage of Annexin V-positive cells was quantified by flow cytometry at different time post-infection. As shown in Panel C, treatment with the pan-caspase inhibitor Z-VADfmk was assessed by quantifying Annexin V staining by flow cytometry. As shown in Panel D, cell lysates were analyzed by immunoblot with anti-caspase 3, anti-caspase 9 and anti-caspase 8 antibodies. As shown in Panel E, mitochondrial membrane potential was analyzed by way of JC-1 staining.

FIG. 3: illustrates that HDIs enhance VSV replication in primary cancer tissues but not in normal tissues and further illustrates that HDIs and VSV synergistically kill ex vivo cultured prostate cancer cells while sparing normal cells. As shown in Panels A and B, ex vivo specimens were inoculated with 5×10⁶ pfu/ml of VSVΔd1-GFP in the absence or the presence of HDI treatments. GFP expression was monitored 48 hours post-viral inoculation. As shown in Panel C, normal PBMCs were isolated from a healthy donor, pre-treated or not with MS-275 or SAHA for 24 hours and then infected or not with VSV-d51-GFP at 10 MOI. VSV replication and apoptosis induction were determined at different times post-infection by FACS measurement of GFP expression and Annexin V-APC staining, respectively. As shown in Panels D and E, epithelial cells were isolated from radical prostatectomy as prostate cancer tissues and their adjacent normal tissues, respectively. Ex vivo primary cultures were pre-treated or not with MS-275 or SAHA for 24 hours and then infected or not with VSV-d51-GFP at 5 MOI. VSV replication and apoptosis induction were determined at different times post infection by FACS measurement of GFP expression and Annexin V-APC staining, respectively.

FIG. 4: illustrates that HDIs may be used so as to increase VSV replication through inhibition of the interferon antiviral response. PC3 cells were either non-treated (NT) or pre-treated with MS-275 or SAHA for 24 hours and then infected or not with VSV-d51-GFP at 0.1 MOI. As shown in Panel A, culture media was assayed by ELISA to detect human IFN-α production at 24 hours post-infection. As shown in Panel B, levels of VSV M protein, IFN beta, IRF-7, and MxA mRNA synthesis were determined by RT-PCR data at 6 hrs, 12 hrs and 24 hrs post-infection. As shown in Panel C, VSV proteins and IRF-3 activation was determined by Western blot analysis. As shown in Panel D, different cell lines were treated with HDIs for 7 hours and then infected with VSV-d51-GFP at 0.1 MOI in the presence or absence of IFN-α treatment (50IU). GFP expression was monitored at 24 hours post VSVΔ51 inoculation.

FIG. 5: illustrates that HDIs augment the viral infection of additional oncolytic viruses, including double deleted vaccinia (VVDD) and herpes simplex virus mutant, HSV-KM100, in various cancer cell lines. Panels A and B show viral infection.

FIG. 6: illustrates that HDIs enhance VSV infection in tumors in vivo. As shown in Panel A, PC3, M14 and HT29 subcutaneous xenograft tumor models were established in nude mice. After tumor growth, the double treated group received MS-275 intraperitoneally at a concentration of 25 mg/kg/day. Four hours post-administering the second HDI dose, all tumors were injected with 1×10⁶ pfu of VSVΔ51-Luc diluted in 50 μl of PBS. The double-treated group continued to receive 25 mg/kg of MS-275 intraperitoneally every 24 hours until sacrificed. Tumors were then harvested and frozen sections were obtained for IHC analysis using anti-VSV antibody. As shown in Panels B and D, subcutaneous 4T1 and SW620 tumors were established in flanks of Balb/c and CD1 nude mice, respectively. For the 4T1 tumor model, three doses of MS-275 were administered intraperitoneally at a concentration of 20 mg/kg every 12 hours. VSV-Luc (1×10⁸ pfu) was introduced intravenously 4 hours following the second MS-275 dose. IVIS pictures were captured at 24, 48 and 80 hours post-VSV injection. In comparison, the double treated group of the SW620 tumor model received five doses of MS-275 intraperitoneally at a concentration of 20 mg/kg given every 12 hours. VSV-Luc (1×10⁷ pfu) was administered intravenously 4 hours post the third MS-275 dose. IVIS pictures were captured at 32, 56 and 130 hours post-VSV injection. As shown in Panels C and E, the efficacy of MS-275, VSV and VSV+MS-275 in treating tumor bearing mice were compared in both the 4T1 as well as the SW620 tumor models. Treatments were initiated once tumors have reached a palpable size of 4×4 mm. As shown in Panel F, an assessment of VSV biodistribution was performed in Balb/c mice at 24 and 72 hours following a single viral intravenous delivery. Biodistribution analysis was performed in the presence or absence of MS-275 treatment. MS-275 treatment protocol was followed as described for Panel B, above. Major organs were harvested, homogenized and tittered on Vero cells. Each histogram bar represents an average of 2 samples.

FIG. 7: illustrates evidence that the intensity of VSV replication in the tumor site is highly dictated by the kinetics of drug and viral administration. As shown in Panel A, the acetylation of H₃ proteins in PC3 tumors was assessed using IHC analysis at 6 and 24 hours following a single intraperitoneal delivery of 30 mg/kg dose. Skin sections were used as normal control. As shown in Panel B, the SW620 tumor model was used to examine the effects of MS-275 treatment on the kinetics of VSV replication at the tumor site. As shown in Panel C, the presence of viral antigen, the induction of active caspase 3, and the microvasculature were assessed in all mice shown in Panel B at day 10 post-viral delivery.

FIG. 8: illustrates evidence that biodistribution of VSV can be monitored via IVIS at 24 and 72 hours post single viral intravenous delivery of 1×10⁸ pfu. A comparison was set between mice treated with VSV alone versus VSV+MS-275 treatment. Three doses of MS-275 were administered at a concentration of 20 mg/kg every 12 hours. In the double-treated group, VSV was administered after the second drug dose.

FIG. 9: illustrates that HDIs inhibit VSV neutralizing antibodies in vivo. As shown in Panel A, Balb/C mice were treated according to a schedule of treatment. As shown in Panel B, blood samples collected at time points defined in Panel A were used to assess VSVΔ51 neutralizing antibody titers. MS 0.1 (grey), MS 0.2 (dark grey) and EtOH (white) represent MS-275 0.1 mg, 0.2 mg and ethanol (30%) control groups respectively. As shown in Panel C, plasma obtained from blood collected at day 7 (with reference to the schedule defined in Panel A) were used to probe for VSV-G specific antibodies by miniblot. Each number indicates one mouse. EtOH=Ethanol treated control, + indicates a known VSV-G specific antibody control.

FIG. 10: illustrates that trichostatin A increases TKA/VGF-deleted vaccinia virus titers and spread in vitro and reduces the number of metastases in an immuno-competent lung metastasis mouse model. Panel A shows representative photomicrographs of B16 mouse melanoma cells that were pre-treated for 3 hours with either trichostatin A (TSA) 0.156 μM or control (DMSO), and then infected with GFP-tagged TK/VGF-deleted vaccinia virus (VVdd) at a multiplicity of infection of 0.1 then incubated for 48 h. As summarized in Panel B, the number of VVdd plaque forming units (pfu)/ml were calculated for B16 cells which were treated as in Panel A but incubated for 72 h. As shown in Panel C, C57BI6 mice were treated according to a schedule of treatment involving the injection of B16-F10-lacZ cells were injected into the tail veins of the mice. As shown in Panel D, the lungs collected on day 14 (with reference to the schedule outlined in Panel C) were fixed and stained using X-Gal and blue-colored metastases were counted. Data were plotted as a mean value of 5 mice per group, error bars represent the standard deviation. * means difference was statistically significant (p<0.05, T-Test) when comparing to PBS treated control as well as to VVdd or TSA single treatments.

FIG. 11: illustrates that SAHA and Apicidin enhance semliki forest virus titers, spread and cytotoxic ability in glioma cell lines. Panel A shows representative photomicrographs of DBT mouse glioma cells pre-treated for 1 hour with either SAHA 5 μM, Apicidin 1 μM or control (DMSO), and then infected with GFP-tagged semliki forest virus (VA7) at a multiplicity of infection (MOI) of 0.01 for 30 hours. Panel B depicts the fraction of viable cells in VA7-infected cells relative to the control cells treated with drugs alone. The data represents the fraction of viable cells in VA7-infected relative to the control cells treated with drugs alone. As represented in Panel C, DBT, CT2A mouse glioma and U251 human glioma cells were treated with HDAC inhibitors as described with reference to Panel A, then infected with VA7 at a MOI of 0.01. After the indicated incubation times, supernatants were collected and titered on vero cells. Data for Panel C is expressed in pfu/ml.

### DETAILED DESCRIPTION

### Therapeutic Formulations

In one aspect, the invention involves administration (including co-administration) of therapeutic compounds or compositions, such as an oncolytic virus or agents that are effective to increase the susceptibility of a tumor cell to oncolytic viral infection in a host. In various embodiments, such agents may be used therapeutically in formulations or medicaments. Accordingly, the invention provides therapeutic compositions comprising active agents, including agents that are effective to increase the susceptibility of a tumor cell to oncolytic viral infection in a host, and pharmacologically acceptable excipients or carriers.

An effective amount of an agent of the invention will generally be a therapeutically effective amount. A “therapeutically effective amount” generally refers to an amount effective, at dosages and for periods of time necessary, to achieve the desired therapeutic result, such as increasing the susceptibility of a tumor cell to oncolytic viral infection in a host. A therapeutically effective amount a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

In particular embodiments, a preferred range for therapeutically effective amounts of HDIs may vary with the nature and/or severity of the patient's condition. It is to be further understood that for any particular subject, specific dosage regimens should be adjusted over time according to the individual need and the professional judgement of the person administering or supervising the administration of the compositions.

A “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, active agents of the invention may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active agent in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In accordance with another aspect of the invention, therapeutic agents of the present invention, such as agents that are effective to increase the susceptibility of a tumor or cancer cell to oncolytic viral infection in a host, may be provided in containers or kits having labels that provide instructions for use of agents of the invention, such as instructions for use in treating cancers.

Use of the present invention to treat or prevent a disease condition as disclosed herein, including prevention of further disease progression, may be conducted in subjects diagnosed or otherwise determined to be afflicted or at risk of developing the condition. In some embodiments, for oncolytic therapy, patients may be characterized as having adequate bone marrow function (for example defined as a peripheral absolute granulocyte count of >2,000/mm³ and a platelet count of 100,000/mm³), adequate liver function (for example, bilirubin<1.5 mg/dl) and adequate renal function (for example, creatinine<1.5 mg/dl).

Routes of administration for agents of the invention may vary, and may for example include intradermal, transdermal, parenteral, intravenous, intramuscular, intranasal, subcutaneous, regional, percutaneous, intratracheal, intraperitoneal, intraarterial, intravesical, intratumoral, inhalation, perfusion, lavage, direct injection, and oral administration and formulation.

Intratumoral injection, or injection into the tumor vasculature is contemplated for discrete, solid, accessible tumors. Local, regional or systemic administration also may be appropriate. For tumors of >4 cm, the volume to be administered may for example be about 4 to 10 ml, while for tumors of <4 cm, a volume of about 1 to 3 ml may be used. Multiple injections may be delivered as single dose, for example in about 0.1 to about 0.5 ml volumes. Viral particles may be administered in multiple injections to a tumor, for example spaced at approximately 1 cm intervals.

Methods of the present invention may be used preoperatively, for example to render an inoperable tumor subject to resection. Alternatively, the present invention may be used at the time of surgery, and/or thereafter, to treat residual or metastatic disease. For example, a resected tumor bed may be injected or perfused with a formulation comprising an oncolytic virus. The perfusion may for example be continued post-resection, for example, by leaving a catheter implanted at the site of the surgery. Periodic post-surgical treatment may also be useful.

Continuous administration of agents of the invention may be applied, where appropriate, for example, where a tumor is excised and the tumor bed is treated to eliminate residual, microscopic disease. Continuous perfusion may for example take place for a period from about 1 to 2 hours, to about 2 to 6 hours, to about 6 to 12 hours, to about 12 to 24 hours, to about 1 to 2 days, to about 1 to 2 weeks or longer following the initiation of treatment. Generally, the dose of the therapeutic agent via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatments of the invention may include various “unit doses.” A unit dose is defined as containing a predetermined-quantity of the therapeutic composition. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁹, 10¹¹, 10¹², 10¹³ pfu and higher. Alternatively, depending on the kind of virus and the titer attainable, one may deliver 1 to 100, 10 to 50, 100 to 1000, or up to about 10⁴, 10⁵, 10⁶, 10⁷, 10⁸, 10⁹, 10¹⁰, 10¹¹, 10¹², 10¹³, 10¹⁴, or 10¹⁵ or higher infectious viral particles (vp) to the patient or to the patient's cells.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as described herein, with reference to the examples and drawings.

### Example 1

### HDI Treatment Enhances VSV Replication and Synergistically Induces Cell Death in VSV-Resistant Cancer Cells

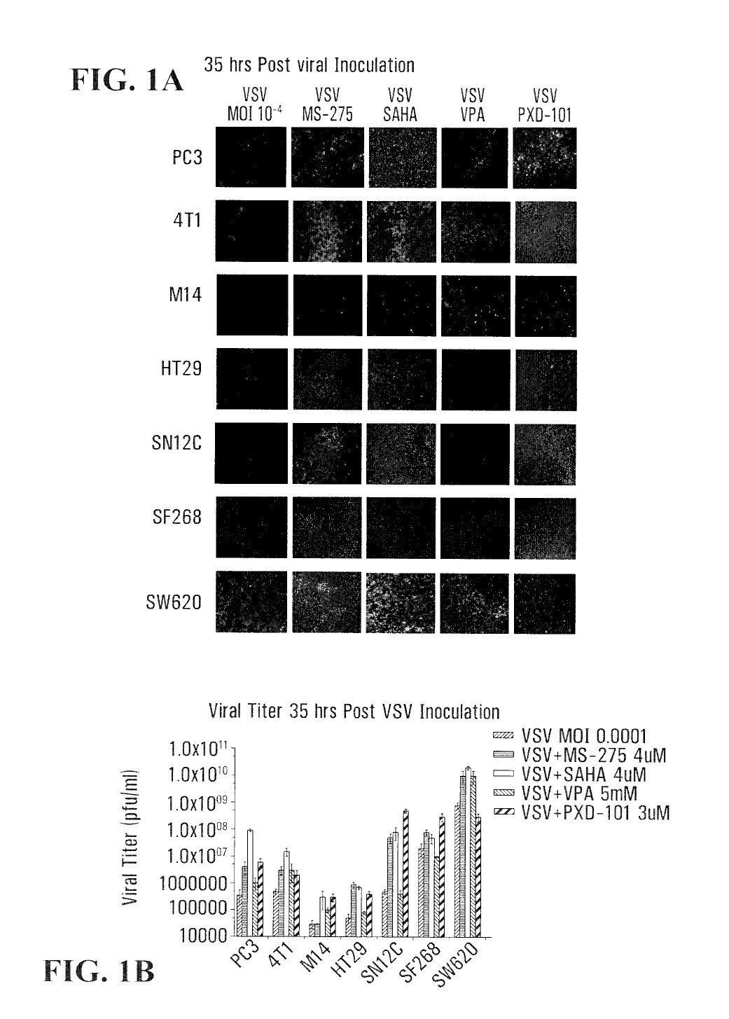

In this Example, the influence of different HDIs such as MS-275, SAHA, VPA and PXD101 were examined on VSV oncolytic potential in different cancer cell lines harboring a relative resistance to VSV infection (PC3 Prostate, 4T1 Breast, M14 Melanoma, HT29 Colon, SN12C Renal, SF268 Central Nervous System, SW620 Colon). To visualize and quantify viral replication in the presence of HDIs, a VSV-d51 strain that expresses the green fluorescent protein (GFP) was used. At significant low MOI of VSV infection (10⁻⁴), treatment with MS-275, SAHA or PXD101 increased the amount of GFP-positive cells as an indication of VSV replication (FIG. 1; Panel A), whereas VPA had little to no effect. These data indicate that the combination of VSV with different classes of HDIs (both hydroxamate and non-hydromate inhibitors) enhances VSV replication in several cancer cell lines and has a greater oncolytic potential than the use of VSV or HDIs alone.

### Example 2

### Combination of VSV and MS-275 or SAHA Synergistically Induces Apoptosis in a Caspase Dependent Manner, Through Activation of the Intrinsic Apoptotic Pathway

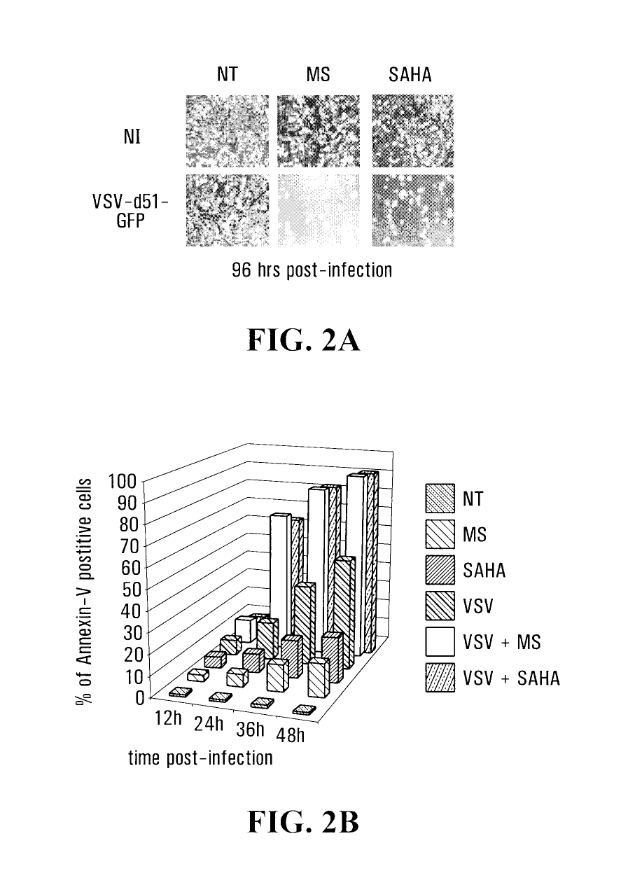

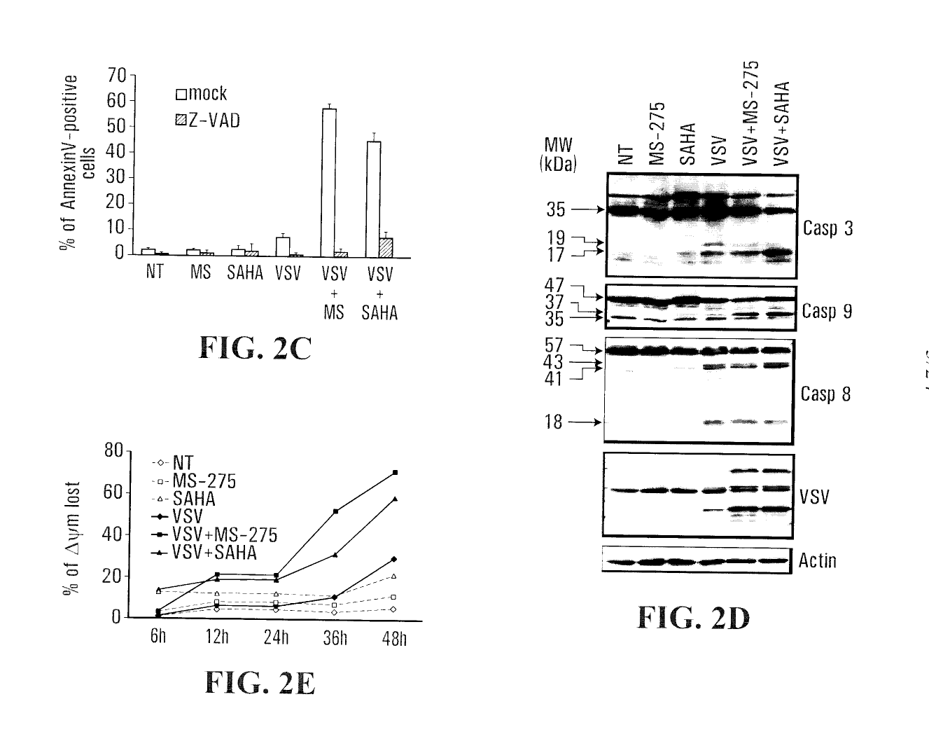

In this Example, induction of apoptosis by the VSV/HDIs combination was investigated in the PC3 prostate cancer model pre-treated with MS-275 or SAHA and infected with VSV-d51. Phase-contrast microscopy pictures showed that only cells receiving the VSV/HDI combination treatment presented morphology of dead cells at 96 hours post-infection whereas VSV-d51, MS-275 or SAHA alone were not able to induce visible signs of cell death (FIG. 2; Panel A). Flow cytometry analyses confirmed that the use of MS-275 or SAHA pre-treatment in combination with VSV-d51 synergistically enhanced the number of Annexin V-positive apoptotic cells (FIG. 2; Panel B). Use of the broad spectrum irreversible caspase inhibitor z-VAD-fmk abrogated activation of apoptosis by VSV+MS-275 or VSV+SAHA, indicating that synergistic induction of cell death by the combination is caspase-dependent (FIG. 2; Panel C). In order to investigate changes of mitochondrial membrane potential, cells were stained with the cationic dye JC-1 and analyzed by flow cytometry. As shown in FIG. 2; Panel D, combination treatment with VSV-d51 and MS-275 or SAHA increased JC-1 green fluorescence in comparison with the use of VSV, MS-275 or SAHA alone, indicating that the VSV/HDI combination triggered apoptosis through the intrinsic mitochondrial pathway. Finally, measurement of caspases 3, 8 and 9 activation by immunoblot assays with antibodies able to detect the activated/cleaved form of these caspases revealed that combination of VSV with HDIs increased cleavage of caspase 3 in comparison with the use of each agent alone and confirmed that synergistic induction of apoptosis by VSV and HDIs is caspase dependent (FIG. 2; Panel E). Moreover, immunoblot assays for detection of caspase 9 showed that significant activation of this caspase was observed only in the presence of the VSV+MS-275 or VSV+SAHA combination treatment (FIG. 2; Panel E). In contrast, while VSV alone was able to induce cleavage of caspase 8, addition of HDI treatment did not increase the level of cleaved caspase 8, indicating that activation of apoptosis by the VSV/HDI combination did not result from enhanced activation of the extrinsic apoptotic pathway (FIG. 2; Panel E). These data therefore revealed that synergistic activation of apoptosis by VSV and HDIs occurred, at least in part, through the mitochondrial apoptotic pathway by synergistic activation of caspase 9.

### Example 3

### HDIs Enhance VSV Spread and Oncolytic Effects in Primary Tumor Specimens while Minimally Affecting the Ability of Normal Tissues to Resist Viral Infection

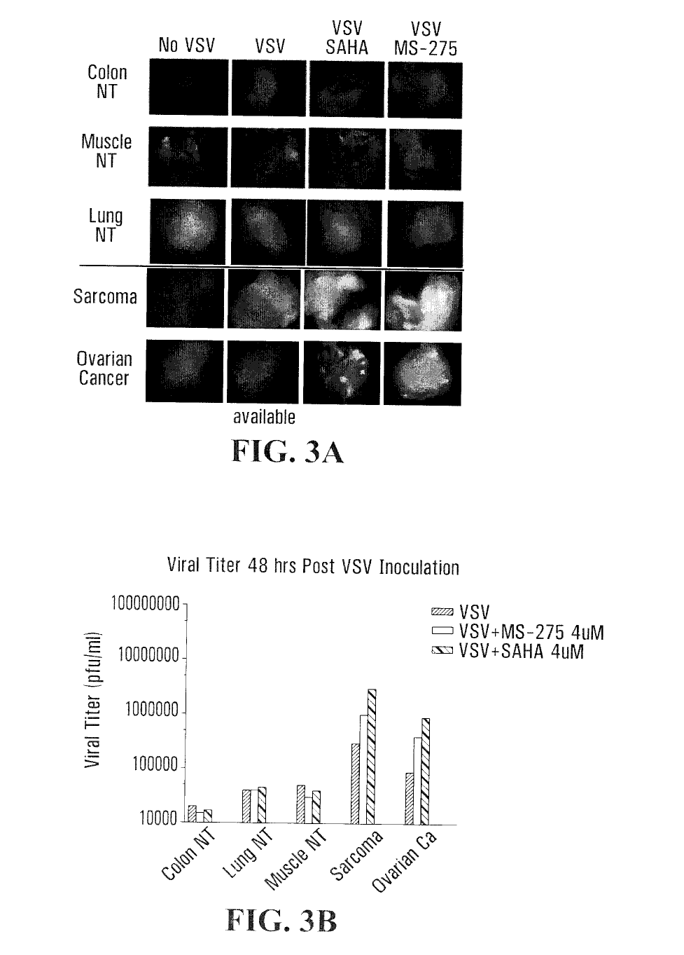

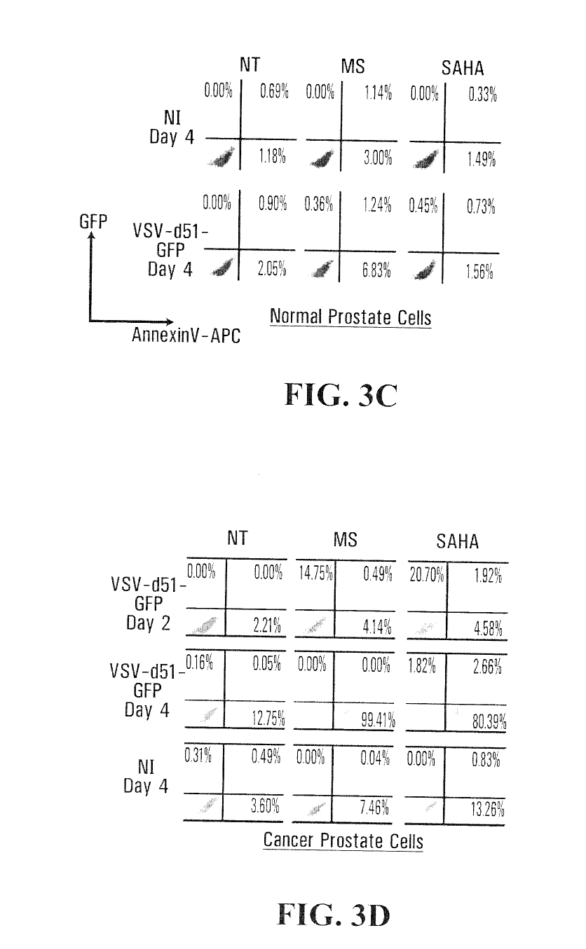

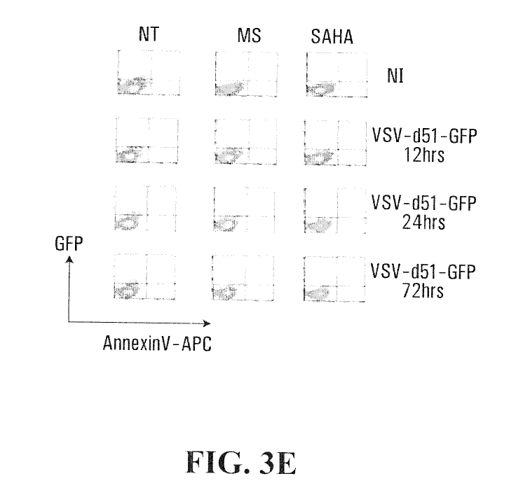

In the present Example, primary samples isolated from cancer (sarcoma, ovarian cancer, prostate cancer) or normal (colon, muscle, lung or prostate) tissues were treated or not with SAHA or MS-275 for 24 hours and then infected or not with VSV-d51-GFP at 5 MOI. At 48 hours post-infection, viral replication was visualized by fluorescent microscopy in order to detect GFP-positive cells. The results indicated that VSV replication was not detectable in primary cancer cells, which indicated their relative resistance to VSV oncolysis; pre-treatment with MS-275 or SAHA allowed effective VSV replication in these cells (FIG. 3; Panel A). This data confirmed the efficacy of the VSV/HDIs combination treatment in primary ex vivo models.

Further, this Example demonstrates that treatment with HDIs did not render normal tissue isolated from colon, muscle, lung or prostate sensitive to VSV infection (FIG. 3; Panel B). These results indicate that the effect of MS-275 and SAHA on VSV replication is specific towards cancer cells. This specificity was further confirmed through the use of PBMCs freshly isolated from healthy donors. Flow cytometry analyses showed that MS-275 or SAHA pretreatment did not increase VSV-GFP replication in normal PBMCS even at high doses of VSV (MOI=10) and importantly that the VSV/HDI combination treatment was not able to induce apoptosis in these cells, as measured by the percentage of Annexin V-positive cells (FIG. 3; Panel C).

In order to examine the efficacy of the VSV/HDIs combination in ex vivo cancer cells, primary prostate cell cultures were established from cancer tissues and their adjacent normal tissues isolated from radical prostatectomy. Flow cytometry analysis for GFP- and Annexin V-positive cells indicated that the level of VSV protein expression was low or undetectable in primary prostate cancer cells whereas pre-treatment with MS-275 or SAHA allowed effective VSV replication in these cells (FIG. 3; Panel D). While HDIs or VSV alone were not able to induce significant cell death, combination of these agents were shown to synergistically induce apoptosis, demonstrating the efficacy of the combination treatment in a primary ex vivo model of prostate cancer (FIG. 3; Panel D). It was also demonstrated that the VSV/HDIs combination had no effect/toxicity on normal prostate cells isolated from the same patient (FIG. 3; Panel E).

### Example 4

### HDIs Enhance VSV Replication in Cancer Cells by Dampening their Innate Antiviral IFN Response

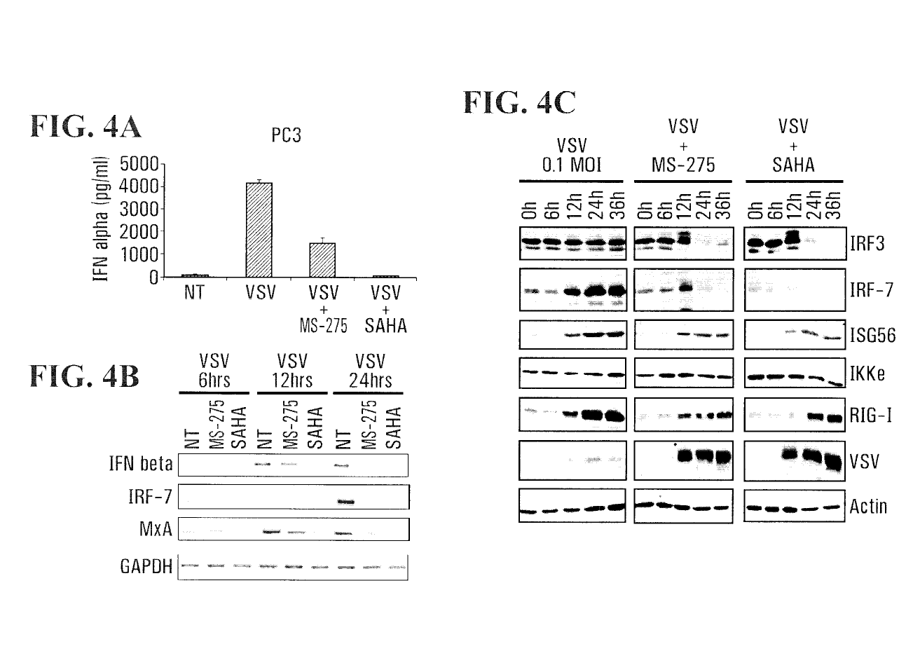

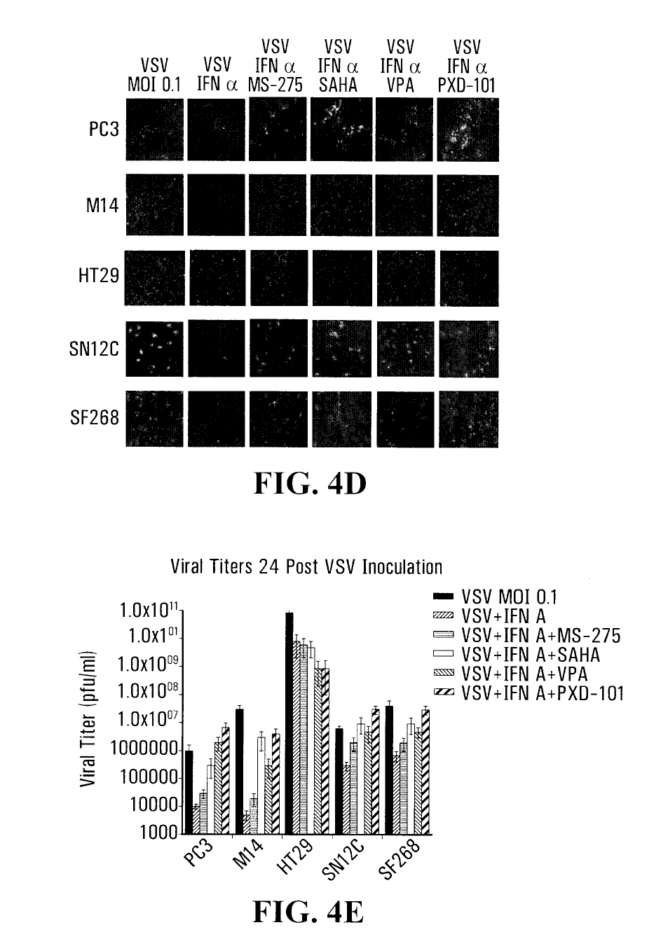

In this Example, PC3 prostate cancer cells, which normally produce a significant level of IFN-α following VSV infection, were pre-treated with either MS-276 or SAHA. It was shown that this pre-treatment significantly inhibited IFN production in the PC3 cells (FIG. 4; Panel A). RT-PCR analysis showed that PC3 cells started to produce IFN-β mRNA at 12 hours post-VSV infection and this production was maintained at 24 hours whereas, in the presence of MS-275 and SAHA, the level of IFN-β mRNA was significantly lower at 12 hours and decreased rapidly to undetectable levels at 24 horrs post-infection (FIG. 4; Panel B). The treatment of PC3 cells with MS-275 or SAHA also decreased the induction of MxA mRNA. It has been shown that MxA is an IFN-inducible gene involved in the control of VSV replication (Schanen, 2006; Schwemmle, 1995) (FIG. 4; Panel B).

Additionally in this Example the influence of HDIs treatment on different steps of the IFN antiviral response pathway was examined by Western blot analysis of cells infected with VSV and either non treated or treated with MS-275 or SAHA (FIG. 4; Panel C). Immunoblot with an anti-VSV antibody confirmed that VSV replication was low in PC3 cells and enhanced in the presence of HDIs pretreatment. In PC3 cells, VSV alone induced expression of IRF7, ISG56 and RIG-I, indicating that VSV infection leads to an activation of the interferon antiviral response. However phosphorylation of IRF3 was not detectable in the presence of VSV alone. When cells were pre-treated with MS-275 or SAHA, enhancement of VSV replication allowed detection of IRF3 phosphorylation and concomitant degradation (Bibeau-Poirier, 2006; Hiscott, 2007; Lin, 1998); the activation of IRF7, ISG56 and RIG-I was inhibited by MS-275 or SAHA treatment. Inhibition of IRF-7 expression by HDIs was confirmed by RT-PCR (FIG. 4; Panel B), indicating that the inhibition occurred at the level of IRF7 transcription. The Western blot analyses indicate that HDIs do not influence the upstream activation pathway of IRF-3 but rather affect IFN production and the establishment of the antiviral response downstream of IRF-3 phosphorylation.

Finally in this Example, different cancer cell lines were treated with IFN-α. IFN-α treatment at the time of viral inoculation was shown to decrease cell permissiveness to viral infection, as shown by monitoring of GFP expression. When HDIs were added to culture media 7 hours prior to IFN-α treatment, cells maintained their permissiveness to VSV infection, indicating that HDIs interfere with the anti-viral effects of IFN-α treatment. The results in this Example indicate that the partial resistance of cancer cells to VSV oncolysis relates to the ability of these cells to mount an effective interferon antiviral response. The data indicates that HDIs may enhance VSV replication in these cancer cells through inhibition of several steps of the interferon antiviral response, from interferon production to response to IFN treatment.

### Example 5

The synergistic effects of HDIs on oncolytic viruses are not limited to that of VSV. VVDD as well as HSV also respond positively to HDI treatment through enhancement of their replication dynamics in a variety of cancer cell lines.

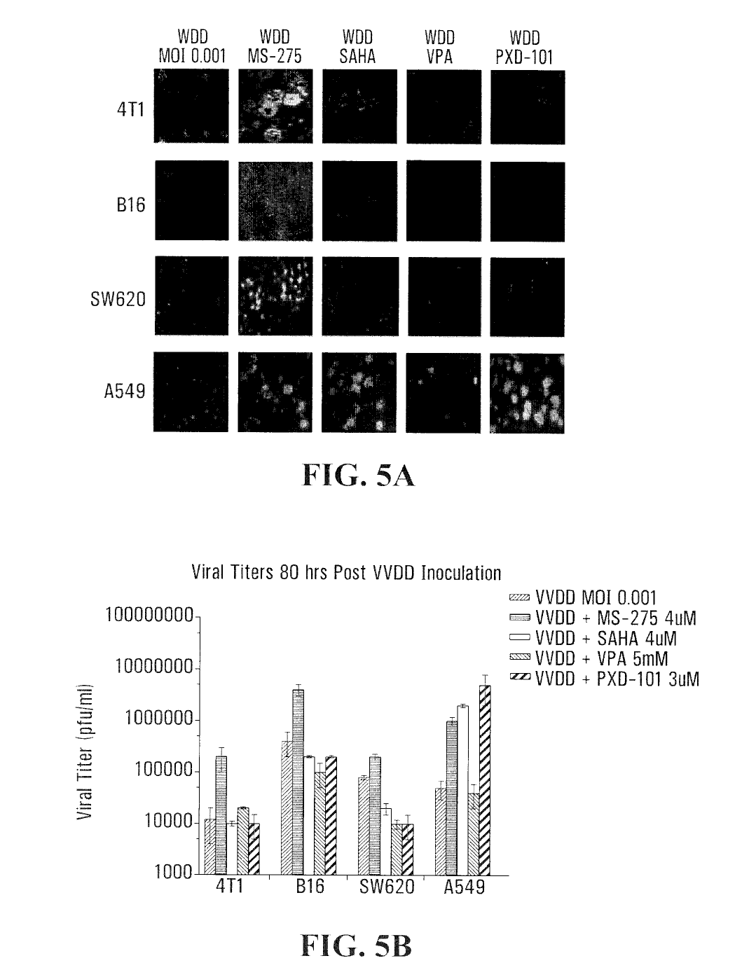

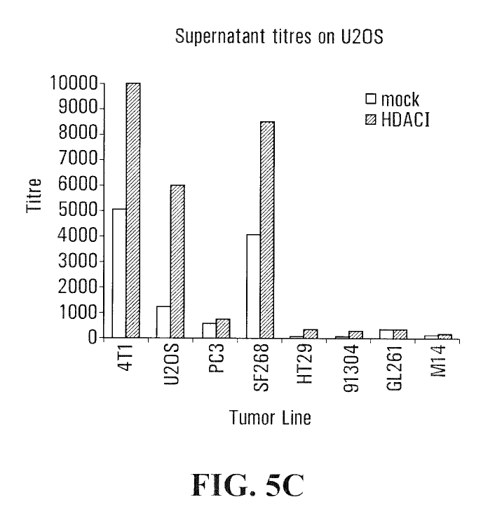

This Example shows, as illustrated in FIG. 5, the synergistic effects of HDIs on the anticancer properties of other oncolytic viral agents such as, the double deleted version of vaccinia virus (vvDD-GFP) (McCart, 2001) as well as the engineered tumor-selective herpes simplex-1 virus (HSV-KM100) (Hummel, 2005). Various cancer cell lines, including PC3, 4T1, HT29, M14, SF 268, A549, SW620, B16 were screened. It was shown that MS-275 was able to synergize the replication of VVDD in 4T1, B16 and SW620 cells. It was demonstrated that VVDD is a slower replicating virus than VSV.

### Example 6

### The HDI MS-275 can be Co-Administered In Vivo to Enhance Specific VSV Replication at the Tumor Sites in Multiple In Vivo Models

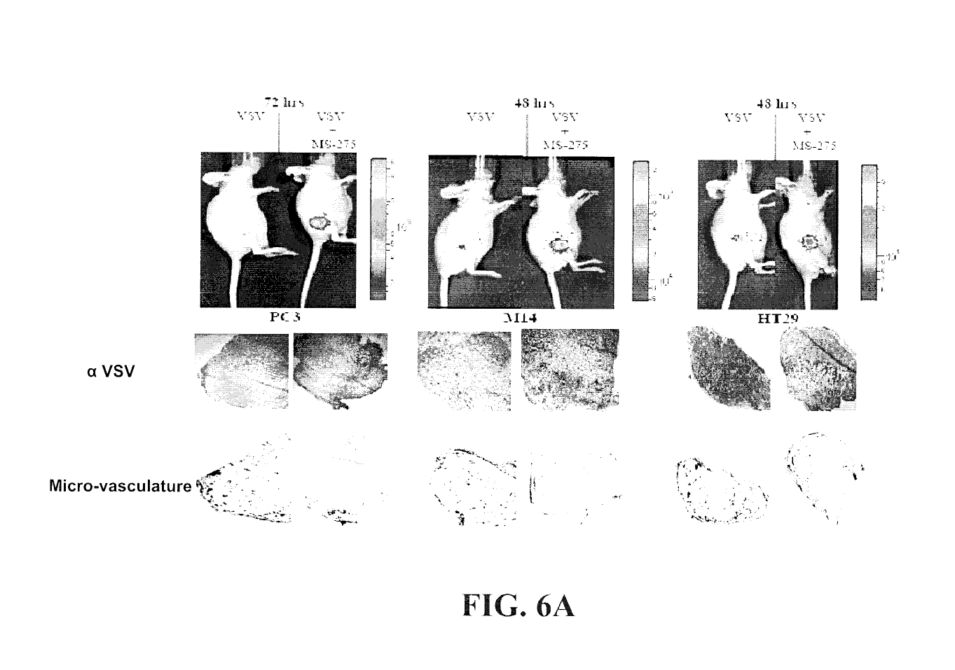

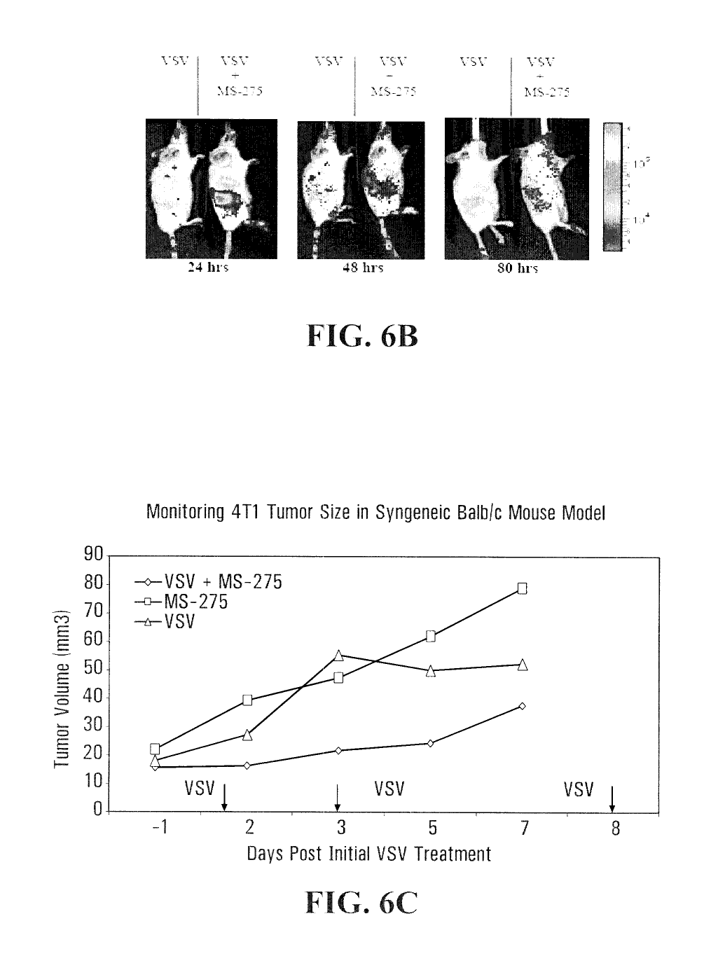

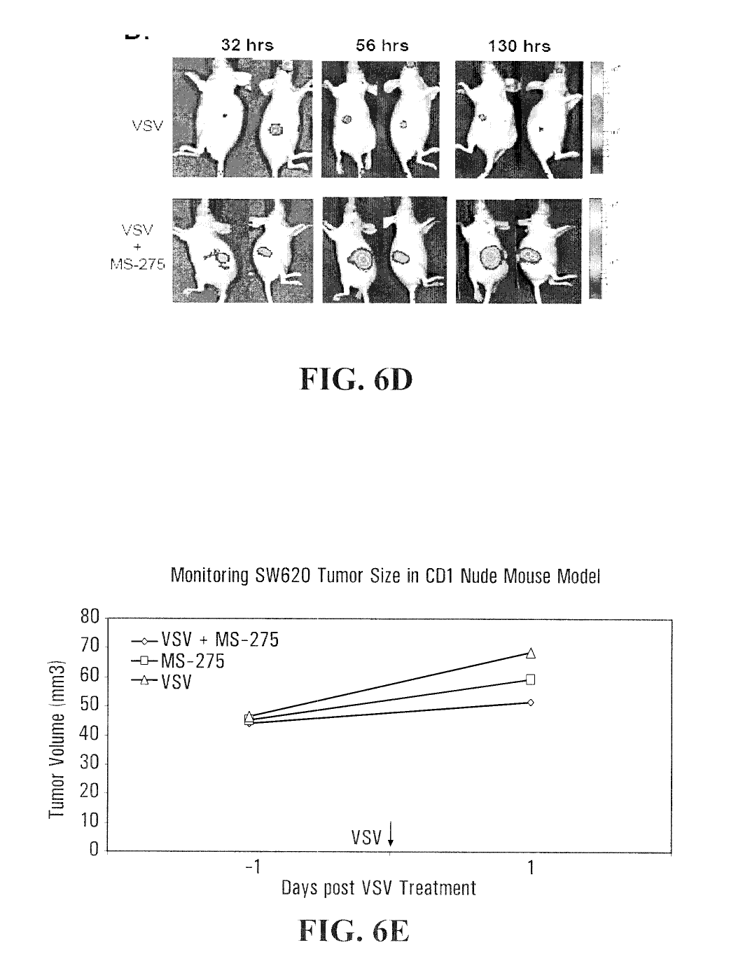

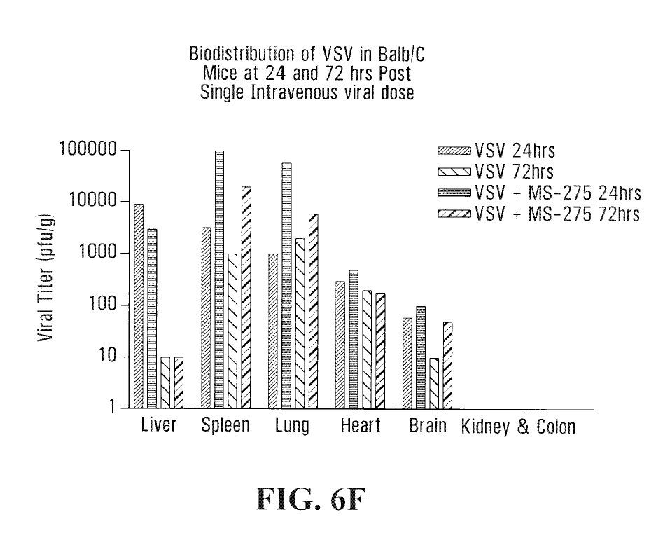

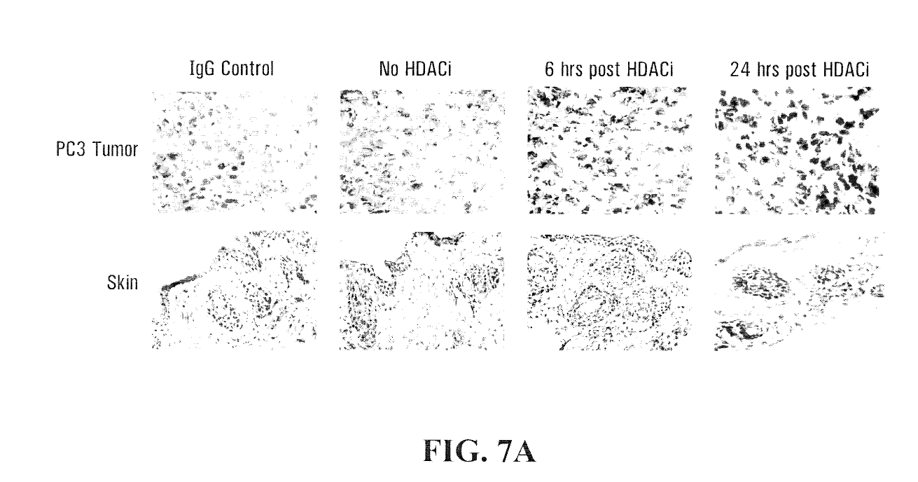

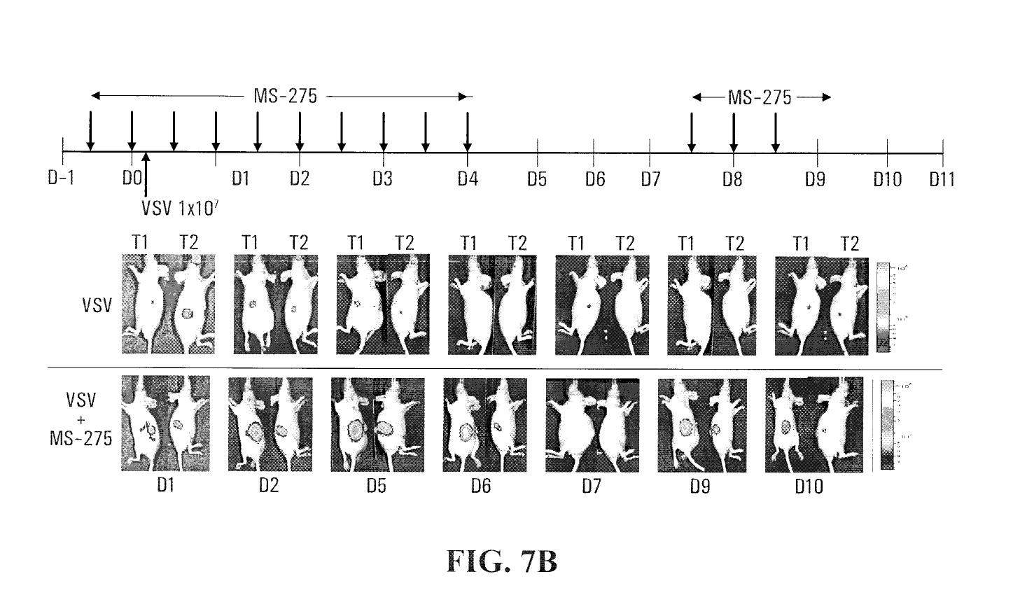

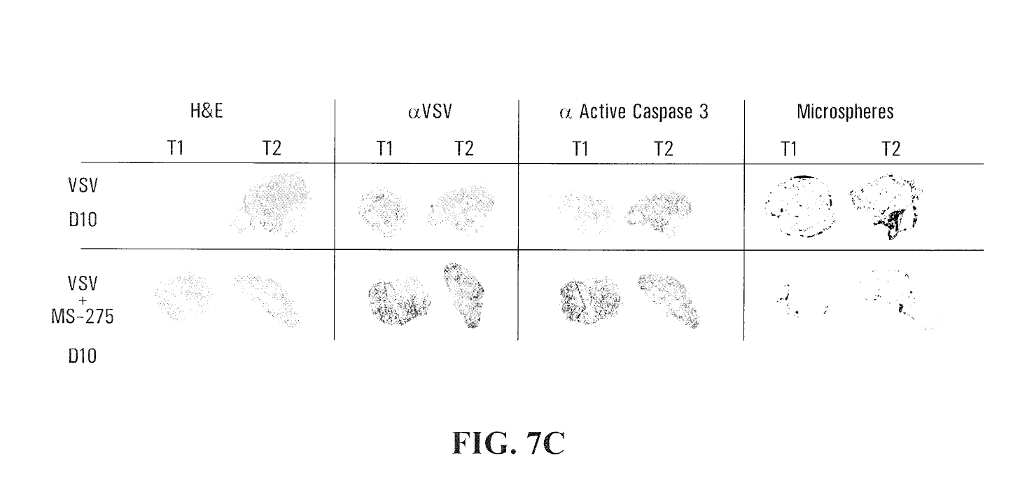

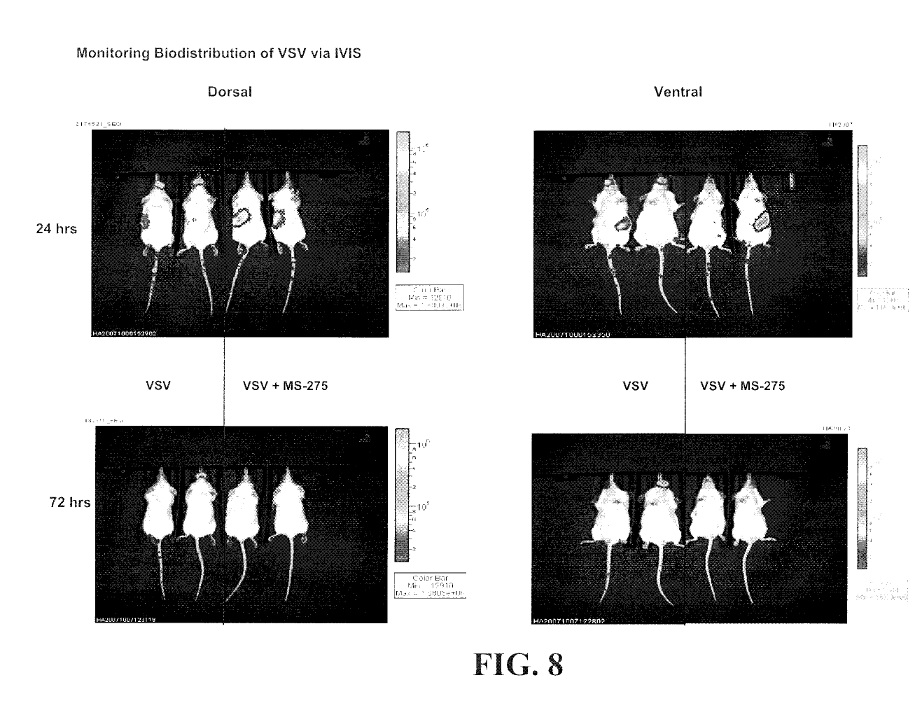

In this Example, three xenograft subcutaneous tumor models were established in CD1 nude mice using PC3, M14 and HT29. In addition, a syngeneic 4T1 subcutaneous tumor model was established in immunocompetent Balb/c mice. It has been shown that these tumor models have poor permissiveness and efficacy profiles after multiple intravenous treatments of VSV alone. The in vivo experiments were performed using VSVΔ51 strain expressing the luciferase gene (VSV-d51-luc). Real time monitoring of viral replication was monitored using In Vivo Imaging System (IVIS), with results illustrated in FIG. 6.

Dosage of drug administration was calculated based on weight. Mice which received intratumoral injection of VSV were treated with an MS-275 dose of 30 mg/kg/day. On the other hand, an MS-275 dose of 20 mg/kg/day. In all scenarios, MS-275 was administered intraperitoneally every 12 hours while VSV was injected 4 hours following the second HDI dose. Using the aforementioned treatment protocols, all of the mice survived the combination treatment. Biodistribution analysis of VSV in Balb/c mice post-MS-275 treatment demonstrated comparable results of viral spread and replication in major organs to the non-MS-275 treated mice. The spleen and lungs were two organs which were sensitive/permissive to VSV in the presence of MS-275 at 24 hours. However, at 72 hours VSV started to clear out of these two organs. This biodistribution data coincided with the mice clinical symptoms where the double-treated group lost approximately 15% of their total weight over the first 72 hours post VSV injection, after which they recovered back to their normal weight.

As illustrated in FIG. 6, pictures captured by IVIS demonstrated a more robust viral replication in tumor-bearing mice that received MS-275 treatment. IHC analysis of frozen sections of the tumors further confirmed more abundant presence of VSV antigen in tumors from animals receiving the VSV/MS-275 combination treatment. The efficacy of the VSV/MS-275 combination with intravenous inoculation of VSVΔ51-Luc was tested and it was demonstrated that, in the presence of MS-275 treatment, this route of viral inoculation is efficient to observe the enhancing effect of HDI on VSV replication in SW620 tumors.

Further in this Example, a model of mammary carcinoma in immunocompetent mice was examined by inoculation of 4T1 cells into the flanks of syngeneic BALB/c mice. When 4T1 tumors developed, mice were treated with MS-275 intra-peritoneally at a concentration of 20 mg/kg/24 hours and with VSVΔ51-Luc introduced intravenously at 4 hours following the second MS-275 dose. IVIS pictures captured at 24, 48 and 80 hours post VSV injection showed a more robust and persistent viral replication in the double-treated mice than in mice treated with VSV alone, again indicating the efficacy of combining MS-275 and VSV.

### Example 7

### The HDI MS-275 can Inhibit VSV Neutralizing and VSV-G Specific Antibody Production in Response to Intravenous Infection with VSV

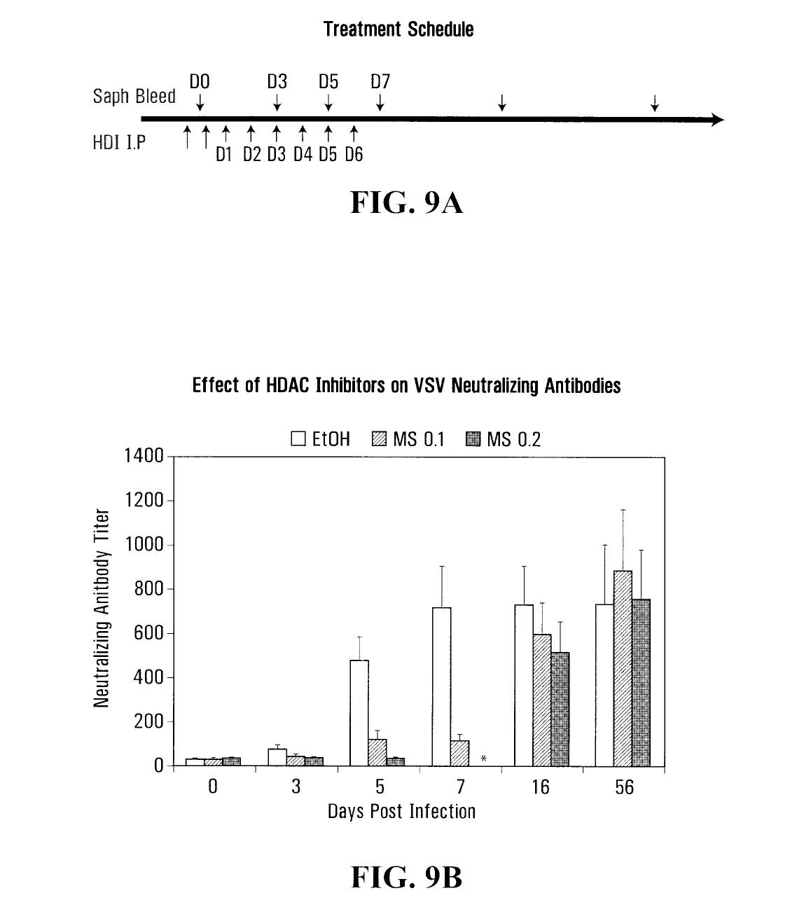

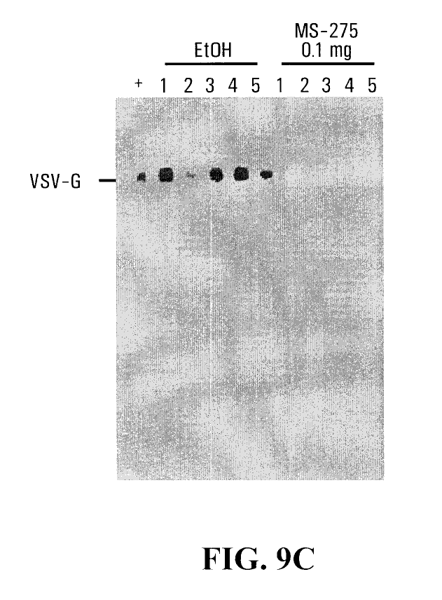

In this Example, Balb/C mice (5 per group) were treated according to a schedule presented in FIG. 9, Panel A. Briefly, mice were first bled (saphenous bleed) then injected intraperitonealy with MS-275 (0.1 or 0.2 mg) or control (Ethanol 30%). 4 hours later, mice were injected with 10⁶ pfu of VSVΔ51 intravenously. Mice were subsequently treated with drugs (or control) daily until day 6 post infection. Blood samples were collected by saphenous bleed on days 3, 5 and 7 post infection. Notably, the group of mice given MS-275 0.2 mg did not receive drug beyond day 5 post-infection due to toxicity concerns nor was any blood collected from these mice on day 7. However, mice had recovered by day 16 at which time blood was collected, and once again at day 56 post infection.

The blood samples were used to assess VSVΔ51 neutralizing antibody titers as shown in FIG. 9, Panel B. Briefly, dilutions of plasma were incubated with 2×10⁵ pfu of VSVΔ51. These were then used to infect vero cells in 96-well plates; 48 hours later alamar blue was used to determine cytopathic effect. Neutralizing antibody titers were determined as being the reciprocal of the dilution of plasma at which 50% of cells were killed by VSVΔ51 (y-axis of FIG. 9, Panel B).

As shown in FIG. 9, Panel C, plasma obtained from blood collected at day 7 was used to probe for VSV-G specific antibodies by miniblot. Briefly, VSV proteins were run on a polyacrylamide gel and transferred on nitrocellulose membrane. Subsequently, a miniblotter was used to incubate the membrane with each plasma sample at 1/100 dilution in non-fat dry milk. Following incubation, peroxidase-linked anti-mouse IgGs were use for chemiluminescent detection.

### Example 8

### Trichostatin A Increases TK/VGF-Deleted Vaccinia Virus Titers and Spread In Vitro and Reduces the Number of Metastases in an Immuno-Competent Lung Metastasis Mouse Model

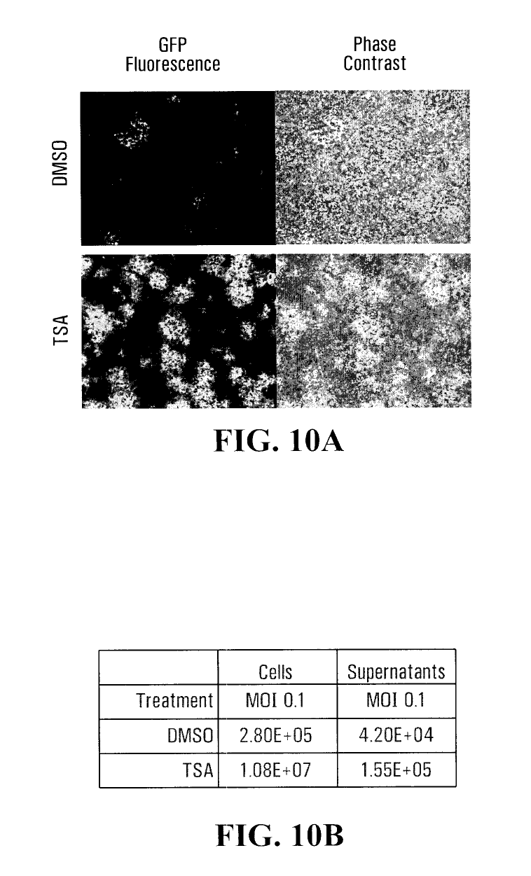

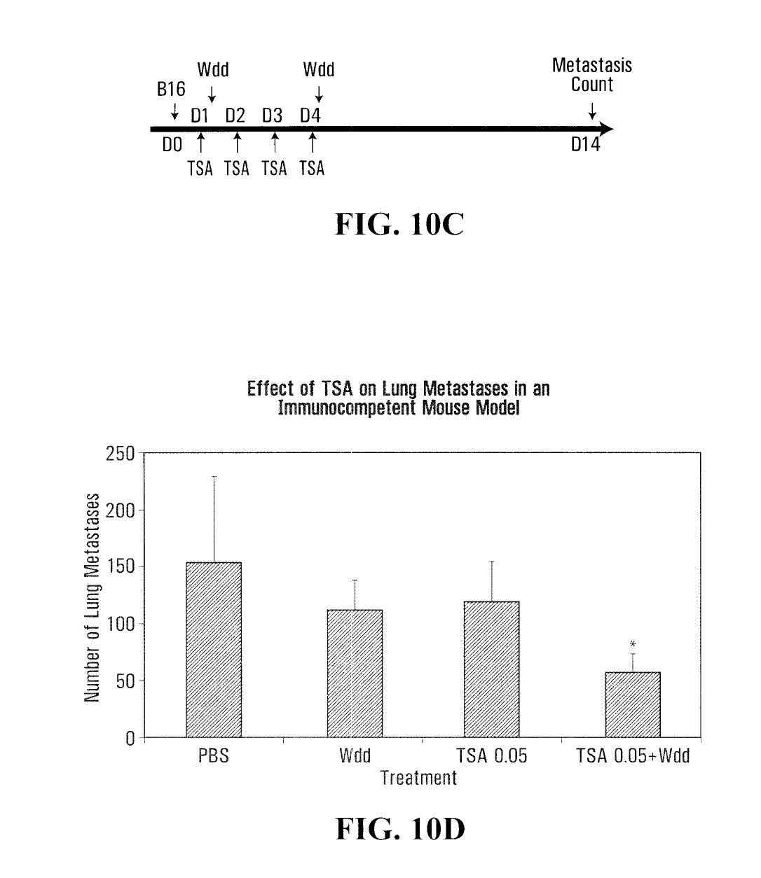

In this Example, B16 mouse melanoma cells were pre-treated for 3 hours with either trichostatin A (TSA) 0.156 □M or control (DMSO) then infected with GFP-tagged TK/VGF deleted vaccinia virus (VVdd) at a multiplicity of infection of 0.1 then incubated for 48 h. Representative photomicrographs were taken under a fluorescence microscope and are shown in FIG. 10, Panel A.

As demonstrated in FIG. 10, Panel B, the supernatants of B16 cells which were treated as described in this Example but for an incubation period of 72 h were collected separately, then lysed by repeated freeze-thaw cycles and tittered on U2OS cells. The numbers compiled in FIG. 10, Panel B indicate VVdd plaque forming units (pfu)/ml.

C57BI6 mice (5 per group) were treated according to a schedule presented in FIG. 10, Panel C. Briefly, on day 0, 10⁵ B16-F10-lacZ were injected in the tail vein. On day 1, mice were treated with 0.05 mg trichostatin A (TSA) or ethanol 30% (control) injected intraperitonealy (i.p); 4 hours later, 10⁷ pfu of VVdd were injected intravenously (i.v). TSA (or control) was subsequently injected i.p daily until day 4, after which a second dose of 10⁷ pfu of VVdd was administered (i.v). On day 14, mice were sacrificed and lungs were collected.

As shown in FIG. 10, Panel, D, lungs collected on day 14 were fixed and stained using X-Gal and blue-colored metastases were counted. Data were plotted as a mean value of 5 mice per group, error bars represent the standard deviation.

### Example 9

### SAHA and Apicidin Enhance Semliki Forest Virus Titers, Spread and Cytotoxic Ability in Glioma Cell Lines

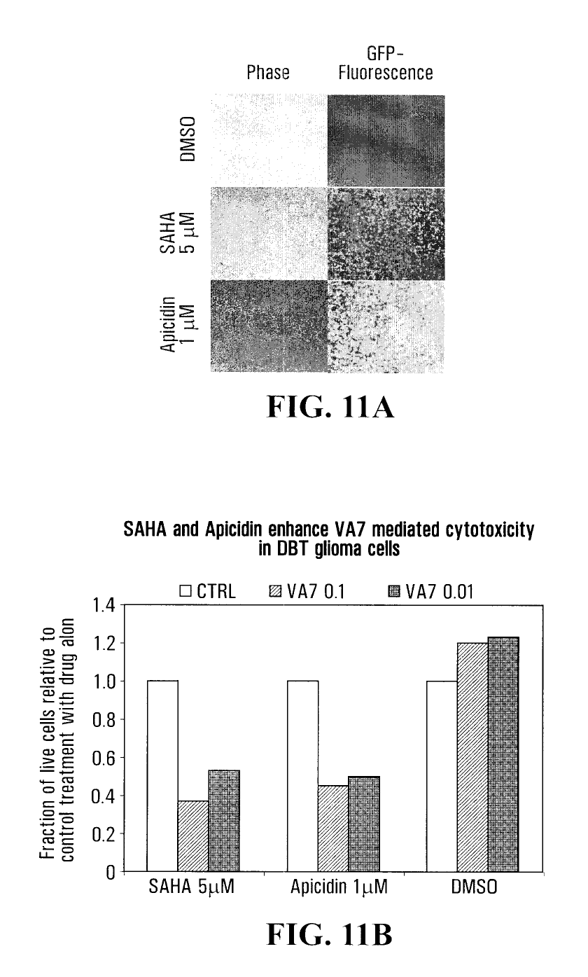

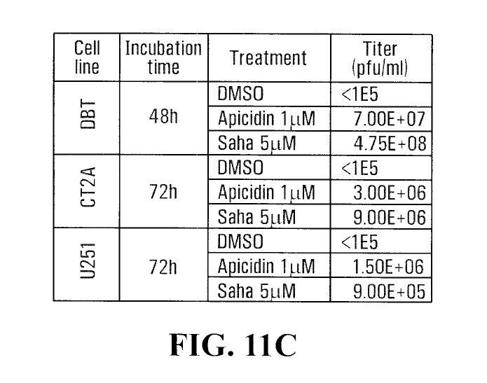

In this Example, DBT mouse glioma cells were pre-treated for 1 hour with either SAHA 5 μM, Apicidin 1 μM or control (DMSO) then infected with GFP-tagged semliki forest virus (VA7) at a multiplicity of infection (MOI) of 0.01. Thirty (30) hours later, photomicrographs were taken using a fluorescence microscope as shown in FIG. 11, Panel A.

As shown in FIG. 11, Panel B, SAHA and Apicidin enhance VA7-mediated cytotoxicity in DBT glioma cells. Briefly, DBT cells were treated with HDAC inhibitors as described above in this Example but for that they were treated with an MOI of VA7 of either 0.1 or 0.01 (as indicated in FIG. 11, Panel B)_and incubated for 48 hours. Thereafter, alamar blue was used to assess cell viability.

As shown in FIG. 11, Panel C, DBT, CT2A mouse glioma and U251 human glioma cells were treated with HDAC inhibitors as described above in this Example and then infected with VA7 at a MOI of 0.01. After the indicated incubation times, supernatants were collected and titered on vero cells. As is shown in Panel C, SAHA and Apicidin enhanced the viral titers compared with the controls (DMSO).

### Methods

### Drugs and Chemicals

For in vitro use, MS-275 (Calbiochem) and SAHA (Alexis Biochemicals) were dissolved in DMSO to a stock concentration of 15 mM and stored at −20° C. For in vivo use, MS-275 was dissolved in PBS, 0.05 N HCl, 0.1% Tween and stored at −20° C. MS-275 or vehicle was delivered as i.p. injections once daily in unanesthetized animals. The pan-caspase inhibitor Z-VAD-fmk was purchased from Calbiochem.

### Viruses

The Indiana serotype of VSV was used throughout this study and was propagated in vero cells (American Type Culture Collection). AV1 VSV is a naturally occurring interferon-inducing mutant of VSV while Δ51 VSV expressing GFP and GFP-firefly luciferase fusion are recombinant interferon inducing mutants of the heat-resistant strain of wild-type VSV Ind. Doubled deleted vaccinia virus expressing GFP was also propagated in vero cells. Virions were purified from cell culture supernatants by passage through a 0.2 μm Steritop filter (Millipore) and centrifugation at 30,000 g before resuspension in PBS (HyClone).

### Cell Lines

PC3 cells were grown in RPMI (Wisent) supplemented with 10% fetal bovine serum (Wisent). SW620 (human colon carcinoma)-derived cells were purchased from American Type Culture Collection and cultured in HyQ Dulbecco's modified Eagle medium (High glucose) (HyClone) supplemented with 10% fetal calf serum (CanSera).

Titration of VSV from Whole Tissue Specimens

Tissue specimens were obtained from consented patients who have under gone resection of their tumors. All tissue specimens were processed within 48 hours post surgical excision. Samples were manually divided using a 15 mm scalpel blade into equal portions under sterile techniques. After the indicated treatment condition, samples were weighed and homogenized in 1 ml of PBS using a homogenizer (Kinematica AG-PCU-11). Serial dilutions of tissue preparations were prepared in serum free media and applied to confluent Vero cells for 45 minutes. Subsequently, the plates were overlayed with 0.5% agarose in media and the plaques were grown overnight. Plaques were counted by visual inspection (between 50 and 200 plaques/plate).

### Flow Cytometry

For measurement of apoptosis, cells were trypsinized, washed in cold PBS and stained on ice with allophycocyanin (APC)-conjugated Annexin V for 15 minutes in Annexin V binding buffer (BD Biosciences). For measurement of mitochondrial membrane depolarization (ΔΨm) cells were trypsinized, washed in PBS and ressuspended in media containing JC-1 (JC-1; CBIC2(3) (5,5′,6,6′-tetrachloro-1,1′,3,3′-tetraethylbenzimidazolyl-carbocyanine iodide-Molecular Probes-Invitrogen Canada Inc.) at final concentration of 1 mM and incubated at 37° C. for 15 min. After incubation cells were subjected to flow cytometry analysis (10⁴ events/measurement) on a FACS Calibur (Becton-Dickinson) and analyzed with FCS Express V3 software.

### IFN ELISA

IFN-α levels were measured using a Human Interferon ELISA kits (PBL Biomedical) per manufacturer's directions. PC3 cells were treated or not with MS-275 (2 μM) or SAHA (5 μM) for 24 hours and then infected with VSV-d51-GFP at 0.1 MOI. One hundred microliters of culture medium was collected at different times post-infection and incubated in a 96-well microtiter plate along with standards supplied by manufacturer. Samples were processed as per manufacturer's instructions and then read on a Dynex plate reader at primary wavelength of 450 nm.

### Western Blotting

Cells were trypsinized, washed in cold PBS and lysed in standard NP-40 lysis buffer. 50 μg of whole-cell extract was run on SDS-polyacrylamide gel and blotted with the following antibodies as indicated: IRF-7 (sc-9083; Santa Cruz), IRF-3 (sc-9082; Santa Cruz), ISG56 (a gift from Ganes Sen) (ref), IKKe (ref), RIG-I (ref), VSV (Polyclonal antiserum to VSV described by Balachandran, 2004), cleaved caspase-3 (cell signaling), cleaved casp 9 (cell signaling), caspase 8 (cell signaling), acetylated histone 3 (Ac-H3) (cell signaling), total H3 (cell signaling), and Actin (sc-8432; Santa Cruz).

### Reverse Transcription and Quantitative Polymerase Chain Reaction.

Total RNA from infected or mock-infected and either HDI-treated or non-treated PC3 cells was isolated as per manufacturer's instruction (RNeasy; Qiagen). 400 ng of RNA was reverse transcribed with Oligo dT primers and 5% of RT was used as template in a Taq PCR. Primers used were as follows: IFN-β forward and reverse; IFN-a forward and reverse, IRF7 forward primer and reverse; VSV, MxA and GAPDH forward and reverse.

### Primary Ex-Vivo Prostate Cancer Cell Cultures

Material was drawn from radical prostatectomy specimens from untreated patients diagnosed with prostate cancer. Prostate cancer tissues and their adjacent normal tissues from radical prostatectomy specimens were obtained from the Sir Mortimer B. Davis-Jewish General Hospital, Department of Urology at McGill University with the collaboration of Dr. T. Bismar under Institutional Review Board approval. For isolation of epithelial cells, prostatic tissue were cut in small pieces and incubated for 45 minutes at 37° C. in culture medium to eliminate blood cells. After washing, pieces were digested in collagenase (2.5 mg/mL), hyaluronidase (1 mg/mL) and deoxyribonuclease (0.01 mg/mL), for 2-3 hours at 37° C. in a shaking water bath. Dispersed stromal cells were separated from digesting fragments and pooled. Resulting tight and large epithelial cell aggregates were washed and further digested with collagenase for another 8-12 hours in the same conditions. Resulting cell aggregates were washed and plated in cell culture plates in Keratinocyte-SFM (Invitrogen) supplemented with manufacturer's serum.

### Isolation of PBMCs

Blood Mononuclear cells were isolated by blood centrifugation (400 g at 20° C. for 25 min) on a Ficoll-Hypaque density gradient (GE Healthcare Bio-Sciences Inc.). PBMCs were cultured in RPMI 1640 supplemented with 15% of heat-inactivated Fetal Bovine Serum (Wisent Inc.) and 100 U/ml penicillin-streptomycin. PBMCs were cultured at 37° C. in a humidified, 5% CO2 incubator.

### Xenograft Cancer Model in Nude Mice

HT29, M14 and SW620 xenograft models were established in 6-8 week old female nu/nu mice obtained from Charles River Laboratories by injecting 1×10⁶ cells in 100 μl PBS subcutaneously in the hind flanks of mice. PC3 xenograft models were established in male nu/nu mice. When tumors reached a palpable size of 3-4 mm, mice were treated either with VSV by either intratumoral, tail vein or intraperitoneal injections or mice were treated with MS-275 by i.p. injections in unanaesthetized animals. After two days of MS-275 treatment, animals were injected with VSV by intratumoral (PC3, HT29, M14) or tail vein injection (SW620). The animals were monitored by IVIS imaging at different time post-VSV injection. Mice were sacrificed at the indicated time points by cervical dislocation and tumors were frozen in Shandon Cryomatrix freezing medium (ThermoElectron, Waltham, Mass.) on dry ice. All experiments were conducted with the approval of the University of Ottawa Animal Care and Veterinary Service. Syngeneic subcutaneous tumors were established by injection of 1×10⁶ cells in 100 μl PBS (SW620) in the left and right hind flanks.)

### Breast Cancer Syngeneic Model in Immunocompetent Mice

Female 6-8-week-old BALB/c immunocompetent mice were obtained from Charles River Laboratories. Syngeneic subcutaneous 4T1 tumors were established by injection of 5×10⁶ cells suspended in 100 μl PBS in the right flanks of mice.

### IVIS Imaging

Mice were injected with D-luciferin (Molecular Imaging Products Company) (200 ml intraperitoneally at 10 mg/ml in PBS) for Firefly luciferase imaging. Mice were anesthesized under 3% isofluorane (Baxter Corp.) and imaged with the In Vivo Imaging System 200 Series Imaging System (Xenogen Corporation). Data acquisition and analysis was performed using Living Image v2.5 software. For each experiment, images were captured under identical exposure, aperture and pixel binning settings, and bioluminescence is plotted on identical color scales.

### Immunohistochemistry (IHC)

Tissues were placed in OCT mounting media (Tissue-Tek) and sectioned in 4 μm sections with a microtome cryostat. Sectioned tissues were fixed in 4% paraformaldehyde for 20 minutes and used for hematoxylin and eosin (H&E) staining or immunochemistry (IHC). IHC was performed using reagents from a Vecastain ABC kit for rabbit primary antibodies (Vector Labs). Primary antibodies used were polyclonal rabbit antibodies against VSV (gift of Earl Brown) and Active Capase3 (BD Pharmingen). Briefly, endogenous peroxidase activity was blocked by incubating with 3% H₂O₂ followed by blocking of non-specific epitopes with 1.5% normal goat serum, then by blocking with avidin and biotin. PBS washes were performed between all blocking and incubating steps. Sections were incubated with either anti-VSV antibody (1:5000; 30 minutes) or anti-Active Caspase3 antibody (1:200; 60 minutes) followed by anti-rabbit biotinylated secondary antibody. The avidin: biotinylated enzyme complex was added and the antigen was localized by incubation with 3,3-diaminobenzidine. Sections were counterstained with hematoxylin. For assessment of cell morphology, sections were stained with hematoxylin and eosin according to standard protocols. Whole tumor images were obtained with an Epson Perfection 2450 Photo Scanner while magnifications were captured using a Xeiss Axiophot HBO 50 microscope.

### REFERENCES

Citation of the following references is not an admission that such references are prior art to the present invention. The following documents are incorporated herein by reference, as if each were specifically and individually indicated to be incorporated by reference herein and as though fully set forth herein:
Bell et al. (2003) Getting oncolytic virus therapies off the ground. Cancer Cell. 4(1): 7-11.
Chang, H.-M. et al. (2004) Induction of interferon-stimulated gene expression and antiviral responses require protein deacetylase activity. Proc Natl. Acad. Sci. U.S.A. 101(26): 9578-9583.
Chiocca, E. A. et al. (2004). A phase I open-label, dose-escalation, multi-institutional trial of injection with an E1B-attenuated adenovirus, ONYX-015, into the peritumoral region of recurrent malignant gliomas, in the adjuvant setting. Mol. Ther. 10, 958-966.
Everts and van der Poel HG (2005). Replication-selective oncolytic viruses in the treatment of cancer. Cancer Gene Ther. February; 12(2):141-161.
Gojo et al. (2007) Phase 1 and pharmacologic study of MS-275, a histone deacetylase inhibitor, in adults with refractory and relapse acute leukemias. Blood. 109(7); 2781-2790.
Harrow, S. et al. (2004). HSV1716 injection into the brain adjacent to tumor following surgical resection of high-grade glioma: safety data and long-term survival. Gene Ther. 11, 1648-1658.
Hirasawa, K. et al. (2003). Systemic reovirus therapy of metastatic cancer in immune-competent mice. Cancer Res. 63, 348-353.
Ichihashi, Y. (1996) Extracellular enveloped vaccinia virus escapes neutralization. Virology 217, 478-485.
Kaufman, H. L. et al. (2005). Targeting the local tumor microenvironment with vaccinia virus expressing B7.1 for the treatment of melanoma. J. Clin. Invest. 115, 1903-1912.
Lorence, R. M. et al. (2003). Overview of phase I studies of intravenous administration of PV701, an oncolytic virus. Curr. Opin. Mol. Ther. 5, 618-624.
Lorence, R. M. et al. (2005). Continuing the interaction between non-clinical and clinical studies. Third International Meeting on Oncolytic Virus Therapeutics: Banff, Alberta (12 Mar. 2005);
Mastrangelo, M. J. et al. (1999) Intratumoral recombinant GM-CSF-encoding virus as gene therapy in patients with cutaneous melanoma. Cancer Gene Ther. 6(5), 409-422.
McCart et al. (2001). Systemic Cancer Therapy with a Tumor-selective Vaccinia Virus Mutant Lacking Thymidine Kinase and Vaccinia Growth Factor Genes. Cancer Research 61, 8751-8757)
Mian et al. (2003) Fully Human Anti-Interleukin 8 Antibody Inhibits Tumor Growth in Orthotopic Bladder Cancer Xenografts via Down-Regulation of Matrix Metalloproteases and Nuclear Factor-{kappa}B Clin. Cancer Res., Aug. 1, 2003; 9(8): 3167-3175.
Myers, R. et al. (2005). Oncolytic activities of approved mumps and measles vaccines for therapy of ovarian cancer. Cancer Gene Ther. 12, 593-599.
Parato et al. (2005). Recent progress in the battle between oncolytic viruses and tumors. Nat Rev Cancer. 5(12): 965-76.
Pecora, A. L. et al. (2002) Phase I trial of intravenous administration of PV701, an oncolytic virus, in patients with advanced solid cancers. J. Clin. Oncol. 20, 2251-2266.
Reid, T. et al. (2001). Intra-arterial administration of a replication selective adenovirus (dl1520) in patients with colorectal carcinoma metastatic to the liver: a phase I trial. Gene Ther. 8, 1618-1626.
Reid et al. (2002). Intravascular adenoviral agents in cancer patients: lessons from clinical trials. Cancer Gene Ther. 9, 979-986.
Ries S J, Brandts C H. (2004) Oncolytic viruses for the treatment of cancer: current strategies and clinical trials. Drug Discov. Today 2004 Sep. 1; 9(17):759-768
Shah et al., (2003). Oncolytic viruses: clinical applications as vectors for the treatment of malignant gliomas. J. Neurooncol. 65, 203-226.
Stojdl, D. F. et al. (2000). Exploiting tumor-specific defects in the interferon pathway with a previously unknown oncolytic virus. Nature Med. 6, 821-825.
Stojdl, D. F. et al. (2003) VSV strains with defects in their ability to shutdown innate immunity are potent systemic anti-cancer agents. Cancer Cell 4, 263-275.
Xia, Z. J. et al. (2004). Phase III randomized clinical trial of intratumoral injection of E1B gene-deleted adenovirus (H101) combined with cisplatin-based chemotherapy in treating squamous cell cancer of head and neck or esophagus. Ai Zheng 23, 1666-1670.

1. A method of amplifying cancer cell-specific oncolytic viral infectivity in a host, comprising:
(a) administering to the host an amount of a histone deacetylase inhibitor (HDI) effective to increase the susceptibility of a cancer cells in the host to oncolytic viral infection; in conjunction with,
(b) infecting cancer cells in the host with one or more strains of oncolytic virus, to provide virally-infected cancer cells, wherein an oncolytic viral infection of a population of the cancer cells is effective to cause apoptosis in virally-infected cancer cells.

2. A method of amplifying cancer cell-specific oncolytic viral infectivity in a host, comprising:
(a) infecting cancer cells in the host with one or more strains of oncolytic virus, to provide virally-infected cancer cells, wherein an oncolytic viral infection of a population of the cancer cells is effective to cause apoptosis in virally-infected cancer cells; in conjunction with,
(b) administering to the host an amount of a histone deacetylase inhibitor (HDI) effective to inhibit production of oncolytic-virus-specific antibodies in the host.

3. The method of claim 1, wherein the HDI is selected from the group consisting of: MS-275, SAHA, VPA, Apicidin, Trichostatin A, and PXD-101.

4. The method of claim 1, wherein the oncolytic virus is selected from the group consisting of: vesicular stromatitis virus (VSV), semliki forest virus, vaccinia virus, and herpes simplex virus, such as HSV1.

5. The method of claim 1, wherein the oncolytic virus is administered to the host systemically.

6. The method of claim 5, wherein the oncolytic virus is administered to the host intravenously.

7. The method of claim 1, wherein the oncolytic virus is administered to the host intra-tumorally.

8. The method of claim 1, wherein the HDI is administered to the host systemically.

9. The method of claim 8, wherein the HDI is administered orally.

10. A composition for treating a tumor in a host, said composition comprising:
(a) a histone deacetylase inhibitor (HDI); and
(b) an oncoyltic virus.

11. The composition of claim 10, wherein the HDI is selected from the group consisting of: MS-275, SAHA, VPA, and PXD-101.

12. The composition of claim 10, wherein the oncolytic virus is selected from the group consisting of: vesicular stromatitis virus (VSV), semliki forest virus, vaccinia virus, and herpes simplex virus, such as HSV1.

13-15. (canceled)

16. The method of 1, wherein the oncolytic virus and the HDI are co-administered to the host.

17. (canceled)

18. The method of claim 1, wherein the host is a human.

19.-24. (canceled)

25. A method of treating a subject with cancer comprising administering to the subject an effective amount of a histone deacetylase inhibitor (HDI) and an effective amount of an oncolytic virus.

26. The method of claim 25, wherein the HDI and the oncolytic virus are co-administered to the subject.

27. The method of claim 25, wherein the subject is human.

28. A method of inhibiting oncolytic virus-specific antibodies in a subject comprising administering a histone deacetylase inhibitor (HDI) to a subject infected with an oncolytic virus.

29. A method of enhancing oncolytic virus infection of a tumor cell comprising contacting the tumor cell with a histone deacetylase inhibitor (HDI) before, during, or after exposure to an oncolytic virus.

30. A method of inhibiting an interferon response of a cancer cell comprising contacting a cancer cell being infected or infected with an oncolytic virus with a histone deacetylase inhibitor (HDI).
